(19) United States
(12) Patent Application Publication
(10) Pub. No.: US 2015/0289749 A1
(43) Pub. Date: Oct. 15, 2015

(54) IMAGING AND TREATMENT DEVICE

(71) Applicant: VOLCANO CORPORATION, San Diego, CA (US)

(72) Inventors: Jeremy Stigall, San Diego, CA (US); Emmett Kearney, San Diego, CA (US)

(21) Appl. No.: 14/644,750

(22) Filed: Mar. 11, 2015

Related U.S. Application Data
(60) Provisional application No. 61/978,354, filed on Apr. 11, 2014.

(51) Int. Cl. A61B 1/00; A61M 25/09; A61B 1/313; A61B 8/00; A61B 8/12; A61N 7/02; A61B 1/018; A61B 5/00

(52) U.S. Cl. CPC A61B 1/00087; A61B 1/018; A61M 25/09; A61B 5/0066; A61B 5/0084; A61B 8/445; A61B 8/12; A61N 7/022; A61B 1/00045; A61B 1/00009; A61B 1/3137; A61B 1/00016; A61B 5/4836; A61N 2007/0078

(57) ABSTRACT

An imaging and treatment device comprises a catheter body, an imaging transducer, and an elongated member. The imaging transducer is disposed at a distal portion of the catheter body. A treatment element is disposed at a distal end of the elongated member, and the elongated member is movable within a lumen of the catheter body.

### RELATED APPLICATIONS

This application claims the benefit of and priority to U.S. provisional application Ser. No. 61/978,354 filed Apr. 11, 2014, the contents of which are incorporated by reference herein in its entirety.

### FIELD OF THE INVENTION

The invention relates to medical devices, systems, and methods for use in, for example, renal denervation.

### BACKGROUND

Hypertension is one of the most prevalent cardiovascular risk factors, afflicting 34% of adults worldwide and is a leading cause of mortality worldwide. Due to noncompliance to pharmacological therapy or resistance to medical therapy, only a small sub-group of afflicted adults have hypertension under control. The renal sympathetic nervous system has been identified as a major contributor to the complex pathophysiology of hypertension, states of volume overload (such as heart failure) and progressive renal disease. Disruption of these renal sympathetic nerves has positive effects on hypertension and other diseases, such as sleep apnea, insulin resistance, and metabolic changes in polycystic ovary syndrome. The renal sympathetic efferent and afferent nerves are positioned within and immediately adjacent to the wall of the renal artery, and have a crucial role in sympathetic nervous system signaling and activation. Thus, the interior lumen of the renal artery is a targeted location for treatment applications and procedures.

Renal sympathetic denervation (RDN) is a method of treatment for diseases such as hypertension and is performed by delivering high frequency energy within the lumen of the renal arteries to disrupt the network of renal afferent and efferent nerves. Commonly, renal denervation procedures involve the delivery of radio frequency (RF) to the interior lumen of the renal artery. For example, once a catheter is positioned in the renal artery, the tissue is treated by applying RF, and each RF application is followed by retraction by at least 5 mm and rotation by 90 degrees of the catheter tip from the first distal main renal artery bifurcation to the ostium. The process is repeated until the nerves are effectively treated.

Visualization of tissues during renal sympathetic denervation procedures requires the application of externally applied imaging modalities, such as fluoroscopy or by venography and angiography. Venography and angiography require the injection of contrast dyes into the patient for visualization of the anatomy of the renal arteries using an externally applied x-ray imaging modality. During this procedure, the patient and the medical staff are exposed to radiation, which can increase the chances of cancer and other radiation concerns. In addition, guiding the catheter and relying on these visualization means can lead to error, including insufficient treatment application or over-treatment.

### SUMMARY

The invention generally relates to medical devices, systems, and methods for providing denervation therapy utilizing a single catheter with both denervation and intraluminal imaging capabilities. When used for renal denervation, the intraluminal imaging capability can provide an accurate, real-time depiction of the target tissue to allow for precise positioning of the denervation assembly relative to the renal afferent and efferent nerves and to assess the progress of the renal denervation procedure. Aside from renal denervation therapy, the devices and systems of the invention are broadly applicable to any ablative procedure, i.e., wherein the energy level within a tissue is altered to affect a therapeutic change.

The invention recognizes that current intraluminal imaging and interventional techniques do not allow for real-time imaging of the internal lumen of the vessel during a treatment procedure. By contrast, devices and systems of the invention utilize an onboard imaging module capable of locating clusters of afferent and efferent nerves during the denervation procedure; providing a more accurate image of the target tissue while eschewing the need for prolonged exposure to the radiation and contrast media found in the imaging techniques currently employed in denervation.

Furthermore, aspects of the present invention reduce the risk of ineffective delivery of treatment due to inaccurate detection and visualization of afferent and efferent nerves in the renal artery. The onboard imaging capabilities allow for real-time imaging of the intraluminal spaces of arteries and the catheter assembly allows for focused delivery of energy to a selected region of interest once visually located. Real-time visualization of the arterial walls allows for precise placement of the catheter assembly, minimizing possible damage to the kidneys and surrounding vessels. After application, the onboard imaging capabilities allow the treated tissue to be analyzed in order to determine if further treatment is needed, thereby preventing excessive application and the risks associated therewith.

Devices according to the invention may include two ultrasound transducer arrays located at two different positions on the device. Intraluminal regions of arteries are imaged with the first transducer array and energy is delivered from the second transducer array. After delivering energy to a region of interest in the artery, the first transducer array provides for visualization and determination if subsequent energy applications are needed. Furthermore, the second transducer array may be localized on a member within the lumen of the device, wherein an actuator can manipulate and control the position of the member.

### BRIEF DESCRIPTION OF THE DRAWINGS

FIG. 1 depicts a catheter assembly including a catheter body, an imaging assembly, and an actuator for manually or automatically controlling an element of the device.

FIG. 2 depicts an imaging catheter system comprising a multi-lumen catheter, an imaging assembly, and a controller to control the imaging and energy delivery elements of the catheter.

FIG. 3 depicts the distal end of an elongated member disposed within the lumen of a catheter body that comprises a treatment element and an imaging assembly is also shown disposed on the catheter body.

### DETAILED DESCRIPTION

The invention generally relates to imaging and treatment devices, systems, and methods for use in, for example, denervation. In general, the invention involves an imaging and treatment device with a treatment element, such as ultrasonic energy, delivering high intensity energy.

Catheter

In certain embodiments, the device is a catheter and configured for intraluminal introduction to a target body lumen, such as the renal artery. The dimensions and other physical characteristics of the catheter bodies will vary significantly depending on the body lumen that is to be accessed. In particular, catheters can be intended for “over-the-wire” introduction when a guide wire channel extends fully through the catheter body or for “rapid exchange” introduction where the guide wire channel extends only through a distal portion of the catheter body. In other cases, it may be possible to provide a fixed or integral coil tip or guide wire tip on the distal portion of the catheter or even dispense with the guide wire entirely. For convenience of illustration, guide wires will not be shown in all embodiments, but it should be appreciated that they can be incorporated into any of these embodiments.

The imaging catheter of the invention comprises an imaging element disposed on the body of the catheter. The imaging element can form or be integrated within the body of the catheter, circumscribe the catheter, placed on a distal end face of the catheter, and/or run along the body of the catheter. The imaging catheter may also include an outer support structure or coating surrounding the imaging elements. Furthermore, the imaging catheter of the invention comprises an elongated member disposed within the lumen of the catheter body. As discussed below, the elongated member can be movable manipulated within the lumen of the catheter body. The elongated member comprises a treatment element for delivering high intensity energy to a tissue or region or interest. Catheter bodies intended for intravascular introduction will typically have a length in the range from 50 cm to 200 cm and an outer diameter in the range from 1 French to 12 French (0.33 mm: 1 French), usually from 3 French to 9 French. Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

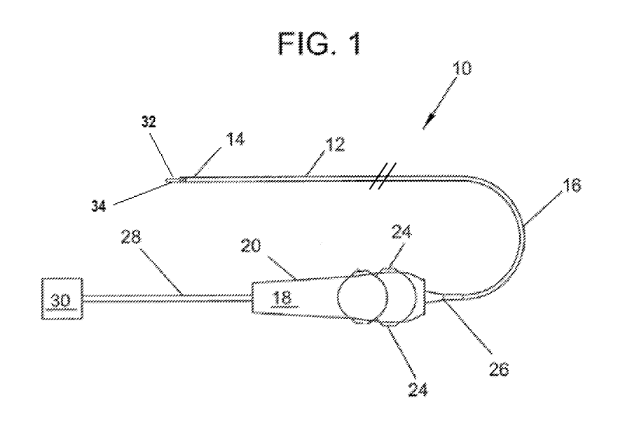

In some embodiments of the invention, the distal portion of the body or catheter of the present invention may have a wide variety of forms and structures. In many embodiments, a distal portion of the catheter comprises transducers for imaging. In some embodiments, the distal portion may be more rigid than a proximal portion, but in other embodiments the distal portion may be equally as flexible as the proximal portion. One aspect of the present invention provides catheters having a lumen. In some embodiments, the lumen of the catheter contains an elongated body that comprising a treatment apparatus or element. In most embodiments a rigid distal portion or housing of the catheter body will have a diameter that generally matches the proximal portion of the catheter body, however, in other embodiments, the distal portion may be larger or smaller than the proximal portion of the catheter. A rigid distal portion of a catheter body can be formed from materials that are rigid or which have very low flexibilities, such as metals, hard plastics, composite materials, NiTi, steel with a coating such as titanium nitride, tantalum, ME-92 (antibacterial coating material), diamonds, or the like. Most usually, the distal end of the catheter body will be formed from stainless steel or platinum/iridium. In some embodiments, elements of the catheter can be manipulated either manually or automatically. In some embodiments, manipulations are accomplished by an actuator in communication with elements of the catheter, as shown in FIG. 1. FIG. 1 illustratively depicts an embodiment of the catheter assembly 10 including a catheter body/shaft 12. The catheter shaft 12 is a generally elongate member having a distal segment 14, a proximal segment 16, and at least one lumen (not shown). The catheter shaft 12 is made, by way of example, of engineered nylon (polyether block amide) and includes a tube or tubing, alternatively called a catheter tube or catheter tubing that has at least one lumen. In some embodiments, an elongated member (not shown) is disposed within the lumen of the catheter body. The proximal segment 16 is attached to a handle 18. The handle 18 includes, by way of example, a housing 20, an actuator 24.

Manipulations of elements in the catheter can be manually or automatically controlled by the actuator. The actuator 24 is manipulated by a user moving an exposed control surface of the actuator 24 (using a finger/thumb) lengthwise along the length of the housing 20 of the handle 18 (as opposed to across the width of the handle 18). In alternative embodiments, thumb-controlled slider actuators replace the rotating knobs. In another alternative embodiment, the actuator is controlled by a computer, or other automatic drivers.

In the illustrative example in FIG. 1, the actuator 24 is accessible (have exposed control surfaces through the housing 20) on two sides of the handle 18. A strain relief 26 protects the catheter shaft 12 at a point where the catheter shaft proximal segment 16 meets the handle 18. A cable 28 connects the handle 18 to a connector 30. The connector 30, which can be any of many possible configurations, is configured to interconnect with an imaging system for processing, storing, manipulating, and displaying data obtained from signals generated by a sensor mounted at the distal segment 14 of the catheter shaft 12.

In one embodiment, the actuator 24 controls the elongated member positioned within the lumen of the catheter body. The user's manipulation of the actuator 24, whether manually or automatically, controls the position of the elongated body by sliding within the lumen, and by rotating the elongated body within the lumen.

In another embodiment, the actuator 24, or another actuator disposed on the housing 20, controls the catheter body. The user's manipulation of the actuator 24, whether manually or automatically, controls the position of the distal end of the catheter.

It should be appreciated that the invention can be used in conjunction with an imaging guide wire, which can be introduced into a lumen of the body to obtain real-time images of the lumen prior to introduction of a catheter. The patient's lumens, into which the guide wire is inserted typically is a lumen of the vasculature. The real-time images obtained may be used to locate a region or location of interest within a body lumen. Regions of interest are typical regions that include a defect or tissues requiring treatment. The invention is also suitable for treating stenosis of body lumens and other hyperplastic and neoplastic conditions in other body lumens, such as the ureter, the biliary duct, respiratory passages, the pancreatic duct, the lymphatic duct, and the like. In addition, the region of interest can include, for example, a location for stent placement or a location including plaque or diseased tissue that needs to be removed or treated.

When a guide wire is used, a catheter according to the invention can be introduced over the guide wire to the intraluminal location of interest. The catheter can obtain images of the intraluminal surface as the catheter moves towards the region of interest, which allows the catheter to be precisely placed into the region of interest and provides for tracking of the catheter along the path of the guide wire. In addition, the catheter can be used to obtain different imaging views of the region of interest. For example, the catheter can be used to locate the renal artery and afferent and efferent nerve clusters found therein.

In certain aspects, the catheter may also serve as a delivery catheter, ablation catheter, extraction catheter or energizing catheter to perform an intraluminal procedure. The catheter may include a treatment element to perform an intraluminal procedure. During the procedure, the catheter may be used to image cross-sections of the luminal surface. In addition, the catheter may have one or more forward and/or distal facing imaging elements to image the luminal space and/or any area in front of or distal to the catheter. After the treatment procedure, the catheter can be removed from the vessel.

A device of the invention may include one or more static imaging assemblies that do not move with respect to the catheter body, or the invention may include one or more moving imaging assemblies. For example, the imaging assembly may be a phased array of ultrasonic transducers for IVUS imaging, or a collection of CCD arrays. An array of elements will typically cover a circumference of the catheter to provide a 360° view of the lumen.

In other embodiments, the imaging assembly may rotate or translate using drive cables within the catheter body. Catheters having imaging assemblies that rotate and translate are known generally as “pull-back” catheters. The principles of pull-back OCT are described in detail in U.S. Pat. No. 7,813,609 and US Patent Publication No. 20090043191, both of which are incorporated herein by reference in their entireties. The mechanical components, including drive shafts, rotating interfaces, windows, and couplings, are similar between the various forms of pull-back imaging.

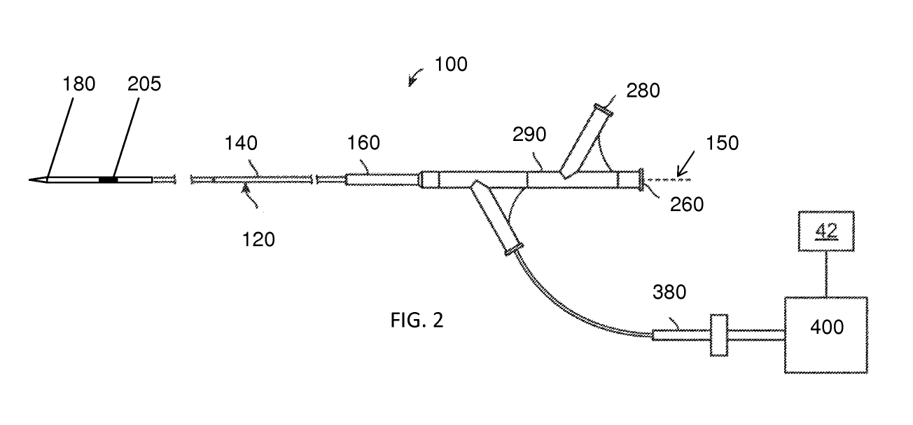

A device of the invention may have multiple lumens. FIG. 2 depicts another embodiment of the invention, wherein the device is configured for multiple lumens. FIG. 2 is merely exemplary, as many other configurations of an imaging catheter system 100 are possible to achieve the principles of the invention. The imaging catheter system 100 includes a catheter 120 having a catheter body 140 with a proximal end 160 and a distal end 180. Catheter body 140 is flexible and defines a catheter axis 150, and may include one or more lumens, such as a guide wire lumen, etc. Catheter 120 also includes an imaging assembly 205 and a housing 290 adjacent proximal end 160. The imaging catheter assembly may comprise any of a number of imaging assemblies. The lumen of the catheter body 140 also comprises an elongated member disposed within the lumen (not shown). In some embodiment, housing 290 includes a connector 280 in fluid communication with the elongated member disposed in the lumen of the catheter body 14. Connectors, such as 260 and 280, may optionally comprise standard connectors, such as Luer-Lok™ (locking mechanisms) connectors.

Housing 290 also accommodates electrical or optoelectrical connectors 380 for powering the imaging assembly and receiving the reflected/scattered light. Connector 380 includes a plurality of electrical connections, each electrically coupled the imaging assembly 205. In some embodiments, the connector 380 is also a mechanical connector in addition to an electrical or optoelectric connector. The mechanical connector can be used to rotate and translate the imaging assembly 205.

A controller 400 may be used to control the imaging and energy delivery. The controller 400 includes a processor, or is coupled to a processor, to control and/or record treatment. The processor will typically comprise computer hardware and/or software, often including one or more programmable processor units running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection, and some or all of the code may also be transmitted between components of the imaging catheter system 100 and within the processor. Controller 400 can connect to imaging systems or computer system through connector 42.

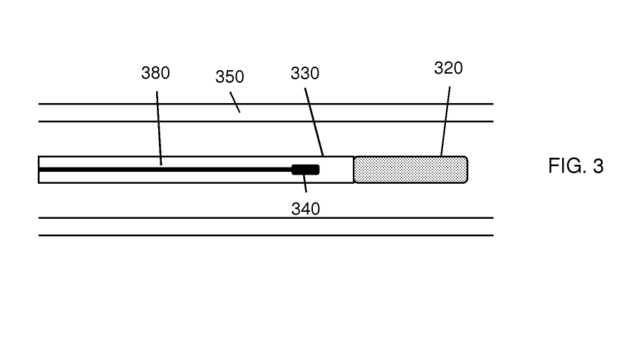

As discussed above, a device of the invention comprises an elongated member 380 disposed within the lumen of the catheter body. FIG. 3 depicts the distal end of the elongated member positioned within the catheter body 330. As shown in FIG. 3, the catheter body 330 comprises an imaging assembly 320, disposed within the intraluminal space of an artery (the cross sectional portion of the artery indicated at 350). As discussed herein, the imaging assembly may comprise a plurality of transducers or a single transducer to image tissues, such as the intraluminal spaces of the renal artery. The elongated member 380 is disposed within the catheter body 330. The treatment element 340 is disposed on the elongated member 380. It should be appreciated that the elongated member 380 and the treatment element 340 can be controlled by an actuator (not shown) to slide and rotate the treatment element within the catheter body 330.

### Imaging Assembly

In certain embodiments, the catheter includes an imaging assembly. Any imaging assembly may be used with devices and methods of the invention, such as optical-acoustic imaging apparatus, intravascular ultrasound (IVUS) or optical coherence tomography (OCT). The imaging assembly is used to send and receive signals to and from the imaging surface that form the imaging data.

In some embodiments, the imaging assembly is an IVUS imaging assembly. The imaging assembly can be a phased-array IVUS imaging assembly, a pull-back type IVUS imaging assembly, including rotational IVUS imaging assemblies, or an IVUS imaging assembly that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein in their entirety.

IVUS imaging is used as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer. Suitable rotational IVUS catheters include, for example the REVOLUTION 45 MHz catheter (offered by the Volcano Corporation).

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receiver sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector.

The transducer subassembly can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLOW (IVUS fluid flow display software offered by the Volcano Corporation).

Suitable phased array imaging catheters include Volcano Corporation's EAGLE EYE Platinum Catheter, EAGLE EYE Platinum Short-Tip Catheter, and EAGLEEYE Gold Catheter. The imaging guide wire of the present invention may also include advanced guide wire designs to include sensors that measure flow and pressure, among other things. For example, the FLOWIRE Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PrimeWire PRESTIGE pressure guide wire, available from Volcano Corp. (San Diego, Calif.), provides a microfabricated microelectromechanical (MEMS) pressure sensor for measuring pressure environments near the distal tip of the guide wire. Additional details of guide wires having MEMS sensors can be found in U.S. Patent Publication No. 2009/0088650, incorporated herein by reference in its entirety.

In addition to IVUS, other intraluminal imaging technologies may be suitable for use in methods of the invention for assessing and characterizing vascular access sites in order to diagnose a condition and determine appropriate treatment. For example, an Optical Coherence Tomography (OCT) catheter may be used to obtain intraluminal images in accordance with the invention. OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable minor, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference minor, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scattered is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

In certain embodiments, angiogram image data is obtained simultaneously with the imaging data obtained from the imaging catheter and/or imaging guide wire of the present invention. In such embodiments, the imaging catheter and/or guide wire may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

One or more imaging elements may be incorporated into an imaging guide wire or imaging catheter to allow an operator to image a luminal surface. The one or more imaging elements of the imaging guide wire or catheter are referred to generally as an imaging assembly. In some embodiments, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

### Denervation Assembly

In a preferred embodiment, the imaging catheter of the invention may be combined with a treatment element. For example, an elongated body is introduced into the lumen of the catheter body and at least a portion of the elongated body is housed within the catheter body or lumen. The elongated body comprises a treatment element, capable of releasing high intensity energy. The elongated member can be for example, a drive cable used in OCT and IVUS systems.

In some embodiments, the treatment element comprises at least one transducer that generates high intensity ultrasound. High-Intensity Focused Ultrasound (HIFU, or sometimes FUS for Focused UltraSound) is a highly precise medical procedure that applies high-intensity focused ultrasound energy to locally heat and destroy diseased or damaged tissue through ablation. HIFU is a hyperthermia therapy, a class of clinical therapies that use temperature to treat diseases. HIFU is also one modality of therapeutic ultrasound, involving minimally invasive or non-invasive methods to direct acoustic energy into the body and at a tissue. In addition to HIFU, other modalities include ultrasound-assisted drug delivery, ultrasound hemostasis, ultrasound lithotripsy, and ultrasound-assisted thrombolysis. Clinical HIFU procedures are typically performed in conjunction with an imaging procedure to enable treatment planning and targeting before applying a therapeutic or ablative levels of ultrasound energy. When Magnetic resonance imaging (MRI) is used for guidance, the technique is sometimes called Magnetic Resonance-guided Focused Ultrasound, often shortened to MRgFUS or MRgHIFU. When diagnostic sonography is used, the technique is sometimes called Ultrasound-guided Focused Ultrasound (USgFUS or USgHIFU). An aspect of the invention allows for HIFU procedures without the need for externally applied imaging modalities.

In one aspect, a treatment element is used to remove an unwanted or damaged vein by delivering energy (RF energy, laser energy, etc.) within a vein to shrink and ultimately close the vein. In some embodiments, the treatment element includes at least one electrode. The electrodes can be arranged in many different patterns along the treatment element. For example, the electrode may be located on a distal end of the elongated member. In addition, the electrodes may have a variety of different shape and sizes. For example, the electrode can be a conductive plate, a conductive ring, conductive loop, or a conductive coil. In one embodiment, the at least one electrode includes a plurality of wire electrodes configured to extend out of the distal end of the imaging electrode.

The proximal end of the treatment element is connected to an energy source that provides energy to the electrodes for delivering high intensity energy. The energy necessary can be provided from a number of different sources including radiofrequency, laser, microwave, ultrasound and forms of direct current (high energy, low energy and fulgutronization procedures). Any source of energy is suitable for use in the treatment element of the invention. Preferably, the source of energy chosen does not disrupt the imaging of the vessel during the procedure with the imaging guide wire and/or imaging catheter.

In operation, the imaging portion of the device can be used to locate a treatment site within the vasculature that requires treatment. Once the treatment site is located, the treatment element is activated in the lumen of the catheter. The electrodes located on the distal end of the elongated member can be positioned and energized by an energy source operably associated with the electrodes. The energized electrodes deliver the energy to the tissue at the treatment site. In one embodiment, the imaging catheter images the luminal surface and lumen during the treatment therapy. In an alternative embodiment, the treatment element deploys several rounds of treatment and the imaging catheter is used to image the treated luminal surface between each round of energy.

In order to minimize risks when performing ablative procedures such as renal denervation (RDN), it is important to monitor and visualize the surrounding tissues. For example, during RDN, the renal artery could be weakened, increasing the chance of embolism, or the renal artery could be perforated or severed. To avoid such damage, prior art devices rely on gated energy delivery to control the temperature of the tissue. That is, RDN devices are programmed to provide predetermined dosing times and wattage based upon accumulated experience and animal/cadaver studies. For example, 4 Watts of radiofrequency energy delivered for 2 seconds has been found to increase the temperature of a cadaver aorta to 65° C. with a particular balloon ablation device. See U.S. Patent Publication No. 2012/0158101 incorporated by reference herein in its entirety. Operation within the suggested range is assumed to provide safe and effective treatment. Nonetheless, without active monitoring of the treatment site, it is impossible to know if the renal artery tissue is being over treated. Using prior art methods, it is impossible to determine if the tissue has been adequately denerved without prolonged blood pressure monitoring after the procedure.

In some aspects, the transducers may comprise capacitive micromachined ultrasonic transducers (CMUTs). CMUTs, which uses micromachining technology, allows for miniaturize device dimensions and produces capacitive transducers that perform comparably to the piezoelectric counterparts. CMUTs are essentially capacitors with one moveable electrode. If an alternating voltage is applied to the device then the moveable electrode begins to vibrate, thus causing ultrasound to be generated. If the cMUTs are used as receivers, then changes in pressure such as those from an ultrasonic wave cause the moveable electrode to deflect and hence produce a measurable change in capacitance. See for example, Ergun et al., Journal of Aerospace Eng., April 2003,16:2(76) page 76-84. CMUT arrays can be made in any arbitrary geometry with very small dimensions using photolithographic techniques and standard microfabrication processes. See Khuri-Yakub et al. J Micromech Microeng. May 2011; 21(5): 054004-054014.

In some aspects, the transducers may comprise piezoelectric micromachined ultrasonic transducers (pMUTs), which are based on the flexural motion of a thin membrane coupled with a thin piezoelectric film. See for example Trolier-McKinstry, Susan; P. Muralt (January 2004). “Thin Film Piezoelectric for MEMS”. Journal of Electroceramics 12 (1-2): 7. doi:10.1023/B:JECR.0000033998.72845.51. It should be noted that pMUTs exhibit superior bandwidth and offer considerable design flexibility, which allows for operation frequency and acoustic impedance to be tailored for numerous applications.

In some embodiments, signals can be transmitted wirelessly, such as by using a transponder. A transponder is a wireless communications, monitoring, or control device that picks up and automatically responds to an incoming signal. The transponder is able to receive signals from transducers on the device of the invention. A transponder is to be understood as a transmitting and receiving unit which upon reception of a wireless electromagnetic signal, transmits a wireless electromagnetic response signal. The device might be part of an infrastructure that consists of base stations, access controllers, application connectivity software, and a distribution system.

In some embodiments, the transponder acts as transmitting and receiving unit which upon reception of a wireless electromagnetic interrogation signal, transmits a wireless electromagnetic response signal. In other embodiments, the transponder receives a wireless electromagnetic signal, and is connected via wire to a processes unit to decode the signal. In a preferred embodiment, at least one transducer is connected to at least one wireless communication unit to transmit signals wirelessly. The transponder is thereby configured to receive the signals of the transducers. See for example U.S. Pat. No. 8,150,449 and U.S. Pat. No. 8,565,202, herein incorporated by reference.

In a preferred embodiment, the device of the invention is positioned in the renal artery of a patient. Using a computer system, the array of transducers located on the catheter body image the interior lumen of the renal artery to thereby display in real time at least a portion of the renal artery on a monitor. The user is able to locate a region of interest and once selected, activate the treatment element of the elongated body to deliver high energy to the region of interest. The user is then able to further view the region of interest to determine whether subsequent applications of energy is needed or required.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An imaging and treatment device comprising:
a catheter body including proximal and distal regions and a lumen disposed within;
an imaging transducer disposed at the distal region of the catheter body; and
an elongated member including a treatment element disposed at a distal region of the elongated member and configured to be movable within the lumen of the catheter body.

2. The imaging and treatment device of claim 1, wherein the catheter body further includes a second lumen for receiving a guide wire.

3. The imaging and treatment device of claim 1, wherein the elongated member is rotatable within the lumen of the catheter.

4. The imaging and treatment device of claim 1, wherein the elongated member is slidable within the lumen of the catheter.

5. The imaging and treatment delivery system of claim 1, wherein the imaging transducer is configured for optical coherence tomography (OCT) or intravascular ultrasound (IVUS).

6. The imaging and treatment device of claim 1, wherein the treatment element comprises at least one transducer.

7. The imaging and treatment delivery system of claim 1, wherein the treatment element delivers high intensity ultrasound energy.

8. The imaging and treatment delivery system of claim 1, further comprising a transponder configured to receive signals from the imaging transducer.

9. A method for treating a tissue, the method comprising:
placing a catheter body proximate to a tissue, wherein the catheter body comprises an imaging transducer and an elongated member, the elongated member comprising a treatment element and being disposed within the lumen of the catheter body;
imaging the tissue with the imaging transducer;
transmitting signals from the imaging transducer to an image processor;
receiving data from the imaging transducer;
processing the data;
locating a region of interest in the tissue;
delivering energy to the region of interest; and
determining if more energy needs to be applied to the tissue.

10. The method of claim 9, wherein processing the data comprises displaying an image of the tissue.

11. The method of claim 9, wherein determining comprises acquiring and comparing at least two images.

12. The method of claim 9, wherein the tissue is a renal artery.

13. The method of claim 9, wherein the energy is high intensity ultrasound energy.

14. The method of claim 9, wherein the transmitting and receiving are done wirelessly.

15. The method of claim 9, wherein the elongated member can be slid and rotated within the lumen of the catheter body.

16. A system for treating a tissue, the system comprising:
a catheter comprising:
a catheter body having proximal and distal regions and a lumen disposed within;
an imaging transducer disposed at the distal region of the catheter body; and
an elongated member including a treatment element disposed at a distal region of the elongated member and configured to be placed slidably within the lumen of the catheter; and
a controller to:
cause the imaging transducer to obtain image data;
receive the data;
process the data; and
release energy from the treatment element.

17. The system of claim 16, wherein the catheter body further includes a second lumen for receiving a guide wire.

18. The system of claim 16, wherein the treatment element comprises at least one transducer.

19. The system of claim 16, wherein the treatment element delivers high intensity ultrasound energy.

20. The system of claim 16, further comprising a transponder configured to receive signals from the imaging transducer.
